(19) United States
(12) Patent Application Publication
(10) Pub. No.: US 2022/0312815 A1
(43) Pub. Date: Oct. 6, 2022

(54) NATURAL FOOD ANTIOXIDANT AND METHOD FOR PREPARING THE SAME

(71) Applicant: Lanzhou Universty, Lanzhou (CN)

(72) Inventor: Quan Liu, Lanzhou (CN)

(21) Appl. No.: 17/281,920

(22) PCT Filed: May 12, 2020
(86) PCT No.: PCT/CN2020/089887

§ 371 Date: Mar. 31, 2021

(30) Foreign Application Priority Data
Dec. 17, 2019 (CN) 201911298544.X

(51) Int. Cl. A23L 33/105; A23L 5/20; A23L 5/30

(52) U.S. Cl. CPC A23L 33/105; A23L 5/23; A23L 5/32; A23V 2002/00

(57) ABSTRACT

The present invention discloses a natural food antioxidant and a method for preparing the same, and belongs to the field of food industry technology. The food antioxidant includes main components of a red clover extract, whose main components are isoflavones, polysaccharides and the mixtures of isoflavones and polysaccharides. Different ingredients have different characteristics in activities, water solubilities and production cost accounting. Combining factors such as activity, solubility and cost, a compounding solution of different ingredients can be used. Especially, the best synergic effect and the highest activity are shown in the mixture of polysaccharides and isoflavones with mass ratio of 1:2. As a leguminous forage, red clover is widely distributed all over the world and has abundant sources. The isoflavones of red clover are usually used as health function. The product of the present invention is easy to be prepared in a safe and environmental process with lower cost, and diversified formulae to satisfy different purposes, which has very wide application prospects.

### FIELD OF THE INVENTION

The present invention relates to the technical field of food industry, particularly to a natural food antioxidant and a method for preparing the same.

### BACKGROUND OF THE INVENTION

A food antioxidant can prevent or slow down the spoilage of food, can prevent the production of a toxic substance in food, the loss of a nutrient and the changes in appearance and color, and can also avoid a plurality of peculiar smells caused by the oxidation of animal fat and oil. A commonly used artificial antioxidant is often added to food. tert-butylhydroquinone (TBHQ), butylated hydroxyanisole (BHA), butylated hydroxytoluene (BHT) and propyl gallate (PG) are widely used. With the improvement of people's health awareness, the safety of a synthetic antioxidant has attracted more and more attentions. It was found that the number of lung cells in a mouse increased after intraperitoneal injection of BHA and BHT in an experiment on the metabolites of a male mouse. Now, the United States, the European Union and other countries have gradually banned the use of such a synthetic antioxidant.

The development and use of a natural antioxidant has become the urgent trend of food science today. Red clover is a perennial leguminous forage, which is widely cultivated worldwide and has abundant resources. It was reported that this plant is rich in isoflavones and has good antioxidative capacities. However, the antioxidative component extracted from the plant of the present invention has better activity than the isoflavone component, better water solubility, and a wider range of adaptability, which has very great development and application prospects.

### SUMMARY OF THE INVENTION

The objective of the present invention is to provide a natural food antioxidant and a method for preparing the same so as to solve the problems raised in the technical background.

In order to achieve the above object, the present invention provides the following technical solutions: a natural food antioxidant, including an extract of red clover extract whose main component is the food antioxidant, the extracts are crude polysaccharide, or crude isoflavone, or refined polysaccharide, or refined isoflavone, or mixtures of refined polysaccharide and refined isoflavone.

Further, the refined polysaccharide and refined isoflavone are prepared with a mass ratio of 1:10-15:1.

Another technical solution provided by the present invention: a method for preparing the natural food antioxidant comprises the following steps:

S1: Preparing a Red Clover Extract:

Collected red clover leaves are deactivated at 105° C. for 2-60 min, are dried at 65° C. for 1-160 h, are crushed, are degreased with a small amount of petroleum ether for 1-10 times, evaporates a solvent, are dried, are weighed with a certain quality of degreased red clover powder, and are ultrasonically extracted by 10-100% ethyl alcohol at 30-70° C. in 10-90 min for 1-50 times. The liquid supernatant is filtered, collected, and combined.

S2: Preparing Crude Polysaccharide and Crude Isoflavone

Crude polysaccharide: extracting solution is concentrated with a vacuum rotator at 15-90° C., when extracting solution is reduced to ⅛ volume, 2-20 times of the volume of absolute ethyl alcohol is slowly added to concentrated solution with slowly stirring. The liquid is put in a refrigerator at 4° C. for overnight. After centrifugation at 4800 rpm for 2-20 min, a brown precipitate is prepared as a crude polysaccharide part, a precipitate part is washed repeatedly with absolute ethyl alcohol for 1-10 times to remove alcohol-soluble impurities, finally, the precipitate is washed with ethyl alcohol and poured into a culture dish, a solvent evaporates to obtain brown red clover crude polysaccharide;

The liquid supernatant is an isoflavone part, and concentrated under reduced pressure to obtain crude red clover isoflavone.

S3: Refining Red Clover Isoflavone:

Pretreated resin is soaked in distilled water for more than 10-90 h. Then the crude isoflavone is dissolved in water, after a sample is loaded and absorbed for 0.5-24 h, 1-5 BV is eluted with distilled water, 30% ethyl alcohol and 50% ethyl alcohol, respectively, and then eluted with 70% ethyl alcohol, and 1-10 BV is finally eluted with 100% ethyl alcohol, 70% ethyl alcohol eluent is collected and concentrated under the reduced pressure to obtain refined isoflavone.

S4: Refining Red Clover Polysaccharide:

Removal of protein: red clover crude polysaccharide is prepared with distilled water to form 1-10 mg/mL solution, added with ¼ volume of sevage solution, shook for 1-30 min, transferred to a separating funnel to keep standing for 1-120 min, and then drained with a solution in a lower layer and protein in a middle layer. This operation is repeated 1-6 times until no protein appears in an organic solvent in the lower layer.

Dialysis: polysaccharide solution from which protein is removed was concentrated at low temperature and reduced pressure, then put into a pretreated dialysis bag, placed in a large-capacity beaker, and filled fully with distilled water, allowed to wait for dialysis, changed with water every 1-16 h and dialyzed for 1-72 h, 1% AgNO3 is used to check residues of NaCl and KCl. Finally the obtained residue is purified polysaccharide.

Further, resin is pretreated: resin is soaked with 20-100% ethyl alcohol for 1-72 h, filtered, rinsed repeatedly with deionized water until there is no alcohol smell and rinsing fluid has no white turbidity. The resin then soaked in 1-30% NaOH for 1-72 h, filtered, rinsed repeatedly with deionized water until pH is neutral, then soaked in 1-30% HCl solution for 1-72 h, filtered and rinsed until the pH is neutral, and dried at 20-90° C. for later use.

Compared with the prior art, the present invention has the following beneficial effects: the present invention proposes the natural food antioxidant and a method for preparing the same. Refined red clover polysaccharide has the advantages of high activity and good water solubility and is firstly considered for use. Secondly, if it is needed to prepare a fat-soluble component, crude red clover polysaccharide or red clover isoflavone can be used. The two types of components have different emphasis in activity. Crude red clover polysaccharide has high antioxidative activity to superoxide anions, but the DPPH activity of isoflavone is better. Considering the cost, comprehensive activity, solubility and the like, the two components can be used for compounding, especially when there is a mass ratio of polysaccharide to isoflavone of 1:2, the synergistic effect of two components is the best, which has the highest activity. The isoflavones of the two components are also popular as health products worldwide. Therefore, a food additive prepared from the plant is not only green and environmentally friendly, but also very safe for the human body, and rich in resources.

### DESCRIPTION OF THE INVENTION

The technical solutions in an embodiment of the present invention will be described clearly and completely below. Obviously, the described embodiments are only a part of embodiments of the present invention, rather than all embodiments. Based on embodiments of the present invention, all other embodiments obtained by the person skilled in the art without creative work shall fall within the protection scope of the present invention.

### Embodiment 1

A natural food antioxidant comprised a main component of a red clover extract. The extract was crude polysaccharide, crude isoflavone, refined polysaccharide and refined isoflavone. Crude polysaccharide and crude isoflavone were prepared based on a mass ratio of 1:2, and refined polysaccharide and refined isoflavones were prepared based on a mass ratio of 1:2.

In order to better demonstrate a process of preparing the natural food antioxidant, this embodiment now proposes a method for preparing the natural food antioxidants, comprising the following steps:

Step 1: Preparing a Red Clover Extract:

Collected red clover leaves were deactivated at 105° C. for 10 min, were dried at 65° C. for 72 h, were crushed, were degreased with a small amount of petroleum ether for 4 times, evaporated a solvent, were dried, were weighed 2 g of degreased red clover powder, and were ultrasonically extracted with 25 times of 40% ethyl alcohol at 50° C. in 30 min for 25 times. The liquid supernatant was were filtered, collected and combined.

Step 2: Preparing Crude Polysaccharide and Crude Isoflavone:

Section 1: crude polysaccharide: extracting solution was concentrated with a vacuum rotator at 45° C. When extracting solution was reduced to ⅛ volume, 4 times of the volume of absolute ethyl alcohol was slowly added to concentrated solution with slowly stirring, to make ethyl alcohol solution reach a concentration of 80%. The liquid was put in a refrigerator at 4° C., and kept standing for overnight. After centrifugation at 4800 rpm for 10 min, a brown precipitate was prepared as a crude polysaccharide part which was washed repeatedly with absolute ethyl alcohol for 3 times to remove alcohol-soluble impurities. Finally, the precipitate was washed with ethyl alcohol and poured into a culture dish. A solvent evaporated to obtain brown red clover crude polysaccharide;

Section 2: the liquid supernatant obtained in step S21 was an isoflavone part and concentrated under reduced pressure to obtain crude red clover isoflavone;

Step 3: Refining Red Clover Isoflavones:

Resin pretreatment: resin was soaked in 95% ethyl alcohol for 24 h, filtered, rinsed repeatedly with deionized water until there was no alcohol smell and the rinsing liquid had no white turbidity, then soaked in 5% NaOH for 24 h, filtered, rinsed repeatedly with deionized water until pH was neutral, soaked with 5% HCl solution for 24 h, filtered and rinsed until the pH was neutral, and dried at 60° C. for later use. Pretreated resin was soaked with distilled water for more than 48 h. Wet-packing resin had a height of 13 cm. 10 g of crude isoflavone was dissolved in water. After a sample was loaded and absorbed for 2 h, 1 BV was eluted with distilled water, 30% ethyl alcohol and 50% ethyl alcohol, respectively. 2 BV was then eluted with 70% ethyl alcohol. Finally, 1 BV was eluted with 100% ethyl alcohol. 70% ethyl alcohol eluent was collected and concentrated under the reduced pressure to obtain refined isoflavone.

Step 4: Refining Red Clover Polysaccharide:

Section 1: removal of protein: red clover crude polysaccharide was prepared with distilled water to form 2 mg/mL solution, added with ¼ volume of sevage solution, shook for 20 min, transferred to a separating funnel to keep standing for 20 min, and drained with a solution in a lower layer and protein in a middle layer. This operation was repeated 3 times until no protein appeared in an organic solvent in the lower layer.

Section 2: dialysis: polysaccharide solution from which protein was removed was concentrated at low temperature and reduced pressure, then put into a pretreated dialysis bag, placed in a large-capacity beaker, and filled fully with distilled water, allowed to stand for dialysis, changed with water every 8 h and dialyzed for 24 h. 1% AgNO3 was used to check residues of NaCl and KCl. Finally purified polysaccharide was obtained.

### Embodiment 2

Antioxidative activity of DPPH: DPPH belonged to a synthetic stable free radical, which has characteristic absorption in a visible light region. A method for measuring DPPH was simple and fast and had good reproducibility.

Flavonoid and a sample of flavonoid and polysaccharide mixed that was prepared in Embodiment 1 were all prepared with 70% ethyl alcohol. The polysaccharide sample, positive control vitamin C (Vc) and vitamin E (Ve) were prepared with distilled water. 1.280 mg/mL stock solution was prepared with each substance to be tested and diluted for 7 concentration gradients for later use.

75 μL of a solution in each concentration to be tested was precisely added to a 96-ferment plate, and then added with 150 μL of 0.1 m MDPPH 70% ethyl alcohol solution. A blank control was set for each reaction hole of each concentration. The blank control was 75 μL of the solution to be tested and 150 μL of 70% ethyl alcohol. The total volume of reaction was 225 μL. The solution was shook well, and kept standing in dark at room temperature for 30 minutes. A light absorption value at a wavelength of 519 nm was measured. A decrease in absorbency indicated an increase in free radical scavenging activity. Each sample was repeated 3 times for each concentration.

Scavenging rate=[A0−(A1−A2)]/A0

A0 was a light absorption value of the DPPH solution plus a solution to be tested at a concentration of zero. A1 was a light absorption value of the solution to be tested that was reacted with DPPH. A2 was a blank control, that is, the light absorption value of each solution to be tested and a blank solvent.

TABLE 1
Antioxidative activity of DPPH

| | IC₅₀ of DPPH(μg/mL) |
|---|---|
| Crude Red Clover | 34.39 + 3.56^{b} |
| Isoflavone | |
| Refined Red Clover | 53.23 ± 1.27^{c} |
| Isoflavones | |
| Crude red clover | 102.33 ± 15.57^{f} |
| polysaccharide | |
| Refined red clover | 51.43 ± 2.72^{c} |
| polysaccharide | |
| 1P: 2IF | 37.49 ± 5.98^{b} |
| 2P: 1IF | 64.87 ± 3.18^{d} |
| 3P: 1IF | 86.11 ± 2.88^{e} |
| Vc | 2.95 + 0.39^{a} |
| Ve | 5.33 + 0.45^{a} |

It should be understood that the terms described in the present invention are only used to describe specific embodiments rather than imposing restriction on the present invention. In addition, for the numerical range referred to in the present invention, it should be understood that it discloses each intermediate value between the upper limit and the lower limit of the range specifically. Furtherm

Note: P represented refined red clover polysaccharide; IF represents refined red clover isoflavone; the refined red clover polysaccharide and refined red clover isoflavone were mixed according to different mass ratios.

A lowercase letter indicated a significant difference (P<0.05).

It could be seen from Table 1 that the activity of refined red clover polysaccharide was equivalent to that of refined isoflavone. The activity is the highest when a mass ratio of refined red clover polysaccharide and refined isoflavone that were mixed was 1:2, and significantly better than each individual component. With the increase of polysaccharide content, the activity of DPPH decreased significantly.

### Embodiment 3

The determination of antioxidative activity of superoxide anions:

O2-• was a free radicals with a longer life in a body. In free radical chain reaction. O2-• acted as an initiator to promote the generation of other free radicals, causing further harm to the body, which was closely related to many diseases. Therefore, the ability of the sample to remove O2-• was usually used as an important indicator of the activity of an antioxidative substance.

Flavonoid and a sample of flavonoid and polysaccharide mixed that was prepared in Embodiment 1 were all prepared with 70% ethyl alcohol. A polysaccharide sample, positive control vitamin C (Vc) and vitamin E (Ve) were prepared with distilled water. 1.280 mg/mL stock solution was prepared with each substance to be tested and then diluted for 7 concentration gradients for later use.

A PMS-NADH-NBT system method was used to determine the activity of scavenging superoxide anions (O2-•). The principle was that reduction type coenzyme I (nicotinamide adenine dinucleotide nad, NADH) was used to reduce phenazine methosulfate (PMS) to produce superoxide anions of O2-•, produced a blue product by reaction with nitro-tetrazolium chloride blue (NBT), and had a strong absorption peak at 560 nm. When being added to the PMS-NADH-NBT system, an antioxidative substance inhibited the reaction of O2-• with NBT. By measuring absorbency, the ability to scavenge free radicals of antioxidant could be calculated. 96-ferment plate was added with 30 μL of NBT (144 μmol/L), 100 μL of a solution to be tested (2.50-125.0 μg/mL, using a solvent to replace a sample solution as a blank), 30 μL of NADH (0.68 mmol/L, prepared with phosphate buffer), 30 μL of newly prepared PMS (60 μmol/L) and 30 μL pH7.4 phosphate buffer. A blank tube was 120 μL buffer and 100 μL solution to be tested, shook for 30 s. A light absorption value of the solution was measured at 560 nm after 5 min.

TABLE 2
The antioxidative activity of superoxide anions

| | IC₅₀ of O²⁻ · (μg/mL) |
|---|---|
| Crude Red Clover | 209.85 ± 7.40^{c} |
| Isoflavone | |
| Purified Red Clover | 219.58 ± 6.95^{d} |
| Isoflavone | |
| Crude Red Clover | 38.30 ± 2.35^{b} |
| Polysaccharide | |
| Refined Red Clover | 2.37 ± 0.03^{a} |
| Polysaccharide | |
| 1P: 2IF | 3.15 ± 0.01^{a} |
| 2P: 1IF | 2.46 ± 0.19^{a} |
| 3P: 1IF | 2.17 ± 0.03^{a} |
| Vc | 208.83 ± 10.41^{d} |
| Ve | 353.33 ± 4.16^{e} |

Note:
P represented refined red clover polysaccharide; IF represented refined red clover isoflavone; the refined red clover polysaccharide and refined red clover isoflavone were mixed according to different mass ratios.

A lowercase letter indicated significant differences (P<0.05).

It could be seen from Table 2 that the activity of red clover isoflavone was equivalent to that of Vc, which was significantly better than Ve, while the activity of red clover polysaccharide was significantly higher than those of Vc and Ve, especially the activity of refined polysaccharide was higher than that of positive control (Vc and Ve) by more than 100 times. The activities of the three compounds were equivalent to those of refined polysaccharide. With the increase of polysaccharide content, the activity was slightly increased.

### Embodiment 4

Evaluation of Interaction of Compounding Components for Antioxidative Activity:

A method (combination index (CI)) for determining a relationship between antioxidative activities of two types of components was used to evaluate the combined antioxidative effect of red clover isoflavone and polysaccharide. Here, Talady and Chou's Median-effect principle combined action index (CI) was adopted.

CI=D1/DX1+D2/DX2

D1 and D2 were action concentrations of flavonoid and polysaccharide, which had the combined inhibitory rate of 50%.

Dx1 and Dx2 were action concentrations at which flavonoid and polysaccharide that acted alone respectively had an inhibitory rate of 50%.

According to the theory of Talady and Chou, if CI=1, it indicated that a combined effect of a drug 1 and a drug 2 was an additive effect. If CI>1, it indicated that the combined effect of the drug 1 and the drug 2 was an antagonistic effect. If CI<1, it indicated that the combined effect of the drug 1 and the drug 2 was a synergistic effect.

TABLE 3
Combined antioxidative index (CI)

| | CI of DPPH | CI of O²⁻· |
|---|---|---|
| 1P: 2IF | 0.71 ± 0.11^{a} | 0.45 ± 0.002^{a} |
| 2P: 1IF | 1.25 ± 0.06^{b} | 0.69 ± 0.053^{b} |
| 3P: 1IF | 1.66 ± 0.06^{c} | 0.69 ± 0.009^{b} |

Note:
A lowercase letter indicated a significant difference (P < 0.05).

It could be seen from Table 3 that when a mass ratio of polysaccharide and isoflavone mixed was 1:2, the two components were synergistic with each other. The synergistic effect on superoxide anions was stronger. But for the activity of DPPH, with the increase of polysaccharide content, polysaccharide and isoflavone had a certain degree of antagonism.

### Embodiment 5

Solubility of Different Preparations in Water

A method for determining water solubility: each test sample was accurately weighed. Each part was 5.0 mg. 5.0 mg of the test sample was added in sequence in a transparent centrifuge tube filled with 1 mL of distilled water at 25° C. of room temperature until precipitation occurred. The operation was repeated. If the test sample could not be completely dissolved after being added, the test sample should stop adding. A vortex could be used to accelerate dissolution. An addition amount of each sample was then recorded in 1 mL of distilled water. A mass of the undissolved part was observed and estimated to obtain an actual dissolved amount of 1 mL of distilled water.

TABLE 4
Dissolution range of different preparation ingredients in water

| | Water solubility (g/L, 25° C.) |
|---|---|
| Crude Red Clover | 5-10 |
| Polysaccharide | |
| Refined Red Clover | 25-30 |
| Polysaccharide | |
| Crude Red Clover | 5 |
| Isoflavone | |
| Refined Red Clover | 5-10 |
| Isoflavone | |

It could be seen from Table 4 that the solubility of polysaccharide in water was stronger than that of isoflavone, and the solubility of a refined sample would increase. Especially, the solubility of refined red clover polysaccharide (greater than 25 g/L) was 5 times as much as that of refined isoflavone and crude polysaccharide.

Using red clover as raw material, different types of natural antioxidant ingredients prepared of the present invention had different functions and application ranges. Therefore, there was a broad market prospect for multi-faceted and multi-purpose application in the development and production of food additives. Refined red clover polysaccharide had the advantages of high activity and good water solubility, and was first considered for use. Secondly, if a fat-soluble component needed to be prepared, crude red clover polysaccharides or red clover isoflavone could be used. The two types of ingredients had different emphasis on activity. Crude red clover polysaccharide had high antioxidative activity to superoxide anions, while the activity of DPPH of isoflavone was better. Thirdly, if considering of cost, comprehensive activity, solubility and other factors, the two components could be used for compounding. Especially, when polysaccharide and isoflavone had a mass ratio of 1:2, the synergy between the two components was the best and the activity was the highest.

In addition, red clover was an excellent pasture, and the isoflavone of red clover was also popular as a health product in the world. Therefore, a food additive prepared from this plant was not only green and environmentally friendly, and very safe for human body, but also rich in resources.

To sum up: for this natural food antioxidant and the method for preparing the same, refined red clover polysaccharide had the advantages of high activity and good water solubility and was firstly considered for use. Secondly, if the fat-soluble component need to be prepared, crude red polysaccharide or red clover isoflavone could be taken into account. The two types of components had different emphasis in activity. Crude red clover polysaccharide had high antioxidative activity to superoxide anions, while the activity of DPPH of isoflavone was better. Considering the cost, the comprehensive activity and solubility and other factors, the two components could be used as the mixture. Especially, when polysaccharides and isoflavone mixed with a mass ratio of 1:2, the mixture had the best synergistic effect and the highest activity. The isoflavones are also popular as the health product worldwide. Therefore, the food additive prepared by using this plant was not only environmentally friendly and very safe to human body but also easy to be prepared because of rich resources.

The above are only preferred specific embodiments of the present invention, but the scope of protection of the present invention is not limited thereto. The person skilled in the art can make equivalent replacements or changes within the technical scope disclosed in the present invention according to the technical solution of the present invention and the inventive concept thereof, which shall fall within the protection scope of the present invention.

1. A natural food antioxidant, characterized in that the main component of the natural food
antioxidants is red clover extract, whose components are crude polysaccharide, or crude isoflavone, or refined polysaccharide, or refined isoflavone, or the mixtures of refined polysaccharide and refined isoflavone.

2. The natural food antioxidant according to claim 1, characterized in that compounds of refined polysaccharide and refined isoflavone are prepared with a mass ratio of 1:10-15:1.

3. A method for preparing a natural food antioxidant according to claim 1, characterized in that the method comprises the following steps:
S1: preparing a red clover extract:
collected red clover leaves are deactivated at 105° C. for 2-60 min, are then dried at 65° C. for 1-160 h, are crushed, are degreased with a small amount of petroleum ether for 1-10 times, are dried again by natural evaporation and ultrasonically extracted by 10-100% ethyl alcohol at 30-70° C. in 10-90 min for 1-50 times, the liquid supernatants are filtered, collected and combined;
S2: preparing crude polysaccharide and crude isoflavone
crude polysaccharide: extracting solution is concentrated with a vacuum rotator at 15-90° C., when extracting solution is reduced to ⅛ volume, 2-20 times of the volume of absolute ethyl alcohol is slowly added to concentrated solution with slowly stirring, the liquid is put in a refrigerator at 4° C. for overnight, after centrifugation at 4800 rpm for 2-20 min, a brown precipitate is prepared as a crude polysaccharide part which is washed repeatedly with absolute ethyl alcohol for 1-10 times to remove alcohol-soluble impurities, finally the precipitate is washed with ethyl alcohol and poured into a culture dish, the brown red clover crude polysaccharide is obtained after natural evaporation of the solvent;
the liquid supernatant is the isoflavone part, and concentrated under reduced pressure to obtain crude red clover isoflavone;
S3: refining isoflavone of red clover:
pretreated resin is soaked in distilled water for more than 10-90 h, then the crude isoflavone is dissolved in water, after a sample is loaded and absorbed for 0.5-24 h, 1-5 BV is eluted with distilled water, 30% ethyl alcohol and 50% ethyl alcohol, respectively, and then eluted with 70% ethyl alcohol, and 1-10 BV is eluted with 100% ethyl alcohol, finally 70% ethyl alcohol eluent is collected and concentrated under the reduced pressure to obtain refined isoflavone;
S4: refining red clover polysaccharide:
removal of protein: the red clover crude polysaccharide is prepared with distilled water to form 1-10 mg/mL solution, added with ¼ volume of sevage solution, shook for 1-30 min, transferred to a separating funnel and kept standing for 1-120 min, and then drained with a solution in a lower layer when protein is in a middle layer, this operation is repeated 1-6 times until no protein appears;
dialysis: polysaccharide solution from which protein has been removed is concentrated at low temperature and reduced pressure, then put into a pretreated dialysis bag, placed in a large-capacity beaker, and filled fully with distilled water, allowed to wait for dialysis, changed with water every 1-16 h and dialyzed for 1-72 h, 1% AgNO3 is used to check residues of NaCl and KCl, finally the obtained residue is purified polysaccharide.

4. The method for preparing the natural food antioxidant according to claim 3, characterized in that the pretreating resin in step S3: the resin is soaked with 20-100% ethyl alcohol for 1-72 h, filtered, rinsed repeatedly with deionized water until there is no alcohol smell and rinsing fluid has no white turbidity, the resin is then soaked in 1-30% NaOH for 1-72 h, filtered, rinsed repeatedly with deionized water until a pH is neutral, finally soaked in 1-30% HCl solution for 1-72 h, filtered and rinsed until the pH is neutral, and dried at 20-90° C. for later use.
